(19) United States
(12) Patent Application Publication
(10) Pub. No.: US 2011/0021572 A1
(43) Pub. Date: Jan. 27, 2011

(54) FUNGICIDE HYDROXIMOYL-TETRAZOLE DERIVATIVES

(76) Inventors: Christian Beier, Saint Genis-Laval (FR); Jurgen Benting, Leichlingen (DE); Isabelle Christian, Lyon (FR); Pierre-Yves Coqueron, Lyon (FR); Christophe Dubost, Lyon (FR); Pierre Genix, Lyon (FR); Benoit Hartmann, Sainte-Foy-Les-Lyon (FR); Daniela Portz, Vettweiss (DE); Arnd Voerste, Koln (DE); Ulrike Wachendorff-Neumann, Neuwied (DE)

Correspondence Address: OSTROLENK FABER GERB & SOFFEN, 1180 AVENUE OF THE AMERICAS, NEW YORK, NY, 100368403, US

(21) Appl. No.: 12/922,672

(22) PCT Filed: Mar. 18, 2009
(86) PCT No.: PCT/EP09/53209

§ 371 Date: Sep. 14, 2010

(30) Foreign Application Priority Data
Mar. 19, 2008 (EP) 08356048.2

(51) Int. Cl. A01N 43/40; C07D 401/12; A01P 3/00; A01P 7/04

(52) U.S. Cl. 514/339; 546/268.4; 514/340

(57) ABSTRACT

The present invention relates to hydroximoyl-tetrazole derivatives, their process of preparation, preparation intermediate compounds, their use as fungicide active agents, particularly in the form of fungicide compositions and methods for the control of phytopathogenic fungi, notably of plants, using these compounds or compositions.

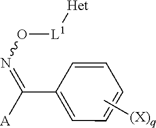

The present invention relates to hydroximoyl-tetrazole derivatives, their process of preparation, preparation intermediate compounds, their use as fungicide active agents, particularly in the form of fungicide compositions and methods for the control of phytopathogenic fungi, notably of plants, using these compounds or compositions.

In European patent application n° 1426371, there are disclosed certain tetrazolyloxime derivatives of the following chemical structure:

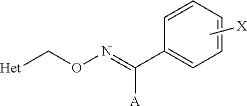

wherein A represents a tetrazolyl group, Het represents either a particular pyridinyl group or a particular thiazolyl group.

In Japanese patent application n° 2004-131392, there are disclosed certain tetrazolyloxime derivatives of the following chemical structure:

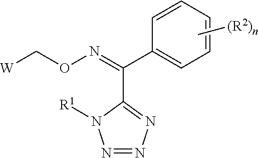

wherein W can be selected in a list of 15 various heterocycle groups.

The compounds disclosed in these two documents do not prove to provide a comparable utility than the compounds according to the invention.

It is always of high-interest in agriculture to use novel pesticide compounds in order to avoid or to control the development of resistant strains to the active ingredients. It is also of high-interest to use novel compounds being more active than those already known, with the aim of decreasing the amounts of active compound to be used, whilst at the same time maintaining effectiveness at least equivalent to the already known compounds. We have now found a new family of compounds which possess the above mentioned effects or advantages.

Accordingly, the present invention provides hydroximoyl-tetrazole derivatives of formula (I):

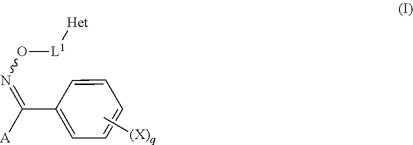

wherein
A represents a tetrazoyl group of formula (A¹) or (A²):

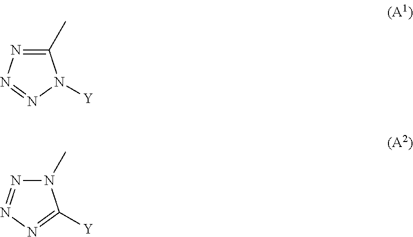

wherein Y represents an alkyl group;
L¹ represents a direct bond or a divalent group selected in the list consisting of
   —(CR¹R²)ₙ— —(CR¹R²)ₘ—C(═O)—(CR¹R²)ₚ—
   —(CR¹R²)ₘ—(CR¹═CR²)—(CR¹R²)ₚ— —(CR¹R²)ₘ—C(═O)—O—(CR¹R²)ₚ
   —(CR¹R²)ₘ—C≡C—(CR¹R²)ₚ— —(CR¹R²)ₘ—O—C(═O)—(CR¹R²)ₚ—
   —(CR¹R²)ₘ—O—(CR¹R²)ₚ— —(CR¹R²)ₘ—C(═O)—NH—(CR¹R²)ₚ—
   —(CR¹R²)ₘ—NH—(CR¹R²)ₚ— —(CR¹R²)ₘ—NH—C(═O)—(CR¹R²)ₚ—
wherein
   n represents 1, 2, 3 or 4;
   m and p independently represent 0, 1, 2 or 3;
   R¹ and R² are independently selected in the list consisting of hydrogen, halogen, [C₁-C₄]-alkyl, [C₁-C₄]-haloalkyl, [C₂-C₄]-alkenyl, [C₂-C₄]-haloalkenyl, [C₂-C₄]alkynyl, [C₂-C₄]-haloalkynyl, [C₃-C₅]-cycloalkyl, [C₃-C₅]-halocycloalkyl, [C₁-C₄]alkoxy, [C₁-C₄]alkoxy-[C₁-C₄]-alkyl, [C₁-C₄]alkoxy-[C₁-C₄]-alkoxy, [C₁-C₄]-haloalkoxy, [C₁-C₄]-haloalkoxy-[C₁-C₄]-alkyl, carbonyloxy, [C₁-C₆]-alkoxy-carbonyl and cyano, R¹ and R² can form a 3- to 7-membered, saturated or unsaturated, carbo- or heterocycle;
Het represents a pyridyl group of formula (Het¹) or a thiazolyl group of formula (Het²);

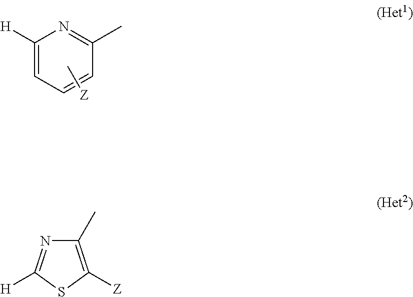

wherein Z represents a an amino group or a group of formula QC(=L)NR— wherein Q represents a hydrogen atom, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy or a substituted or non-substituted phenyl group; L represents an oxygen atom or a sulphur atom; and R represents a hydrogen atom, a substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms;
X independently represents a hydrogen atom, a halogen atom, an alkyl group, an alkoxy group, a cyano group, a methanesulfonyl group, a nitro group, a trifluoromethyl group or an aryl group;
q represents 1, 2, 3, 4 or 5, provided that if q represents 2, 3, 4 or 5 then X represents a halogen atom;
as well as salts, N-oxides, metallic complexes and metalloidic complexes thereof.

Any of the compounds according to the invention can exist as one or more stereoisomers depending on the number of stereogenic units (as defined by the IUPAC rules) in the compound. The invention thus relates equally to all the stereoisomers and to the mixtures of all the possible stereoisomers, in all proportions. The stereoisomers can be separated according to the methods which are known per se by the man ordinary skilled in the art.

Notably, the stereostructure of the oxime moiety present in the tetrazolyloxime derivative of formula (I) includes (E) or (Z) isomer and these stereoisomers form part of the present invention.

According to the invention, the following generic terms are generally used with the following meanings:
halogen means fluorine, chlorine, bromine or iodine;
heteroatom can be nitrogen, oxygen or sulphur;
unless indicated otherwise, a group or a substituent that is substituted according to the invention can be substituted by one or more of the following groups or atoms: a halogen atom, a nitro group, a hydroxy group, a cyano group, an amino group, a sulphenyl group, a pentafluoro-⁶-sulphenyl group, a formyl group, a substituted or non-substituted carbaldehyde O—(C₁-C₈-alkyl)oxime, a formyloxy group, a formylamino group, a carbamoyl group, a N-hydroxycarbamoyl group, a formylamino group, a (hydroxyimino)-C₁-C₆-alkyl group, a C₁-C₈-alkyl, a tri(C₁-C₈-alkyl)silyl-C₁-C₈-alkyl, C₁-C₈-cycloalkyl, tri(C₁-C₈-alkyl)silyl-C₁-C₈-cycloalkyl, a C₁-C₈-halogenoalkyl having 1 to 5 halogen atoms, a C₁-C₈-halogenocycloalkyl having 1 to 5 halogen atoms, a C₂-C₈-alkenyl, a C₂-C₈-alkynyl, a C₂-C₈-alkenyloxy, a C₂-C₈-alkynyloxy, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkylsulphenyl, a C₁-C₈-halogenoalkylsulphenyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a N—C₁-C₈-alkyloxycarbamoyl, a C₁-C₈-alkoxycarbamoyl, a N—C₁-C₈-alkyl-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, substituted or non-substituted C₁-C₈-alkoxycarbonylamino, substituted or non-substituted C₁-C₈-halogenoalkoxycarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₈-alkylaminosulfamoyl, a di-C₁-C₈-alkylaminosulfamoyl, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, (benzyloxyimino)-C₁-C₆-alkyl, C₁-C₈-alkoxyalkyl, C₁-C₈-halogenoalkoxyalkyl having 1 to 5 halogen atoms, benzyloxy, benzylsulphenyl, benzylamino, phenoxy, phenylsulphenyl or phenylamino;
the term “aryl” means phenyl or naphthyl.

In the tetrazoyl group of formula (A¹) or (A²), Y represents an alkyl group. Among these alkyl groups, an alkyl group having 1 to 3 carbon atoms such as a methyl group, an ethyl group, a n-propyl group or an isopropyl group is preferred. Among these alkyl groups, a methyl group or an ethyl group is particularly preferred.

Preferred compounds of formula (I) according to the invention are those wherein L¹ represents a direct bond or a divalent group selected in the list consisting of
—(CR¹R²)ₙ— —C(═O)—(CR¹R²)ₚ—
—(CR¹R²)ₘ—O— —(CR¹R²)ₘ—C(═O)—O—
—(CR¹R²)ₘ—NH— —(CR¹R²)ₘ—C(═O)—NH—
—(CR¹R²)ₘ—C(═O)— —(CR¹R²)ₘ—NH—C(═O)
   wherein
n represents 1 or 2;
m and p independently represent 0 or 1;
R¹ and R² independently represent a hydrogen atom, a halogen atom, a cyano group, substituted or non-substituted C₁-C₈-alkyl, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

More preferred compounds of formula (I) according to the invention are those wherein L¹ represents a direct bond or a divalent group selected in the list consisting of —(CR¹R²)—, —C(═O)—(CR¹R²)— and —C(═O)—; wherein R¹ and R² are independently selected in the list consisting of hydrogen, halogen, methyl, ethyl, isopropyl, trifluoromethyl, difluoromethyl, allyl, ethynyl, propargyl, cyclopropyl, methoxy, trifluoromethoxy and cyano.

Other preferred compounds of formula (I) according to the invention are those wherein X represents a hydrogen atom; a chlorine atom; a fluorine atom; an alkyl group having 1 to 4 carbon atoms, for example a methyl group, an ethyl group, a n-propyl group, an isopropyl group, a n-butyl group, an isobutyl group, a sec-butyl group and a tert-butyl group, in particular a methyl group or a tert-butyl group; an alkoxy group having 1 to 3 carbon atoms, for example a methoxy group, an ethoxy group, a propoxy group and an isopropoxy group, in particular, a methoxy group or an ethoxy group; or a phenyl group, a 4-methylphenyl group and a 4-chlorophenyl group, in particular a phenyl.

Other preferred compounds of formula (I) according to the invention are those wherein q represents 1 or 2. More preferably, q represents 1.

Other preferred compounds of formula (I) according to the invention are those wherein Q represents a hydrogen atom, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₃-C₈-alkynyloxy or a substituted or non-substituted phenyl group.

More preferred compounds of formula (I) according to the invention are those wherein Q represents a methyl group, an ethyl group, a n-propyl group, an isopropyl group, a 1,1-dimethylpropyl group, a n-butyl group, an isobutyl group, a sec-butyl group, a tert-butyl group, an isoamyl group, a 1-methylbutyl group, a 2-methylbutyl group, an neopentyl group, a 1-ethylpropyl group, a n-pentyl group, a hexyl group, a heptyl group and an octyl group, a chloromethyl group, a difluoromethyl group, a trifluoromethyl group, a difluorochloromethyl group, a pentafluoroethyl group, a 3,3,3-trifluoro-n-propyl group and a 1-chlorohexyl group, a cyclopropyl group, a cyclobutyl group, a cyclopentyl group and a cyclohexyl group, a methoxy group, an ethoxy group, a propoxy group, an isopropoxy group, a 1,1-dimethylpropoxy group, a butoxy group, an isobutoxy group, a sec-butoxy group, a tert-butoxy group, an isopentyloxy group, a 1-methylbutoxy group, a 2-methylbutoxy group, an neopentyloxy group, a 1-ethylpropoxy group, a n-pentyloxy group, a hexyloxy group, a heptyloxy group and an octyloxy group, a cyclopropyloxy group, a cyclobutyloxy group, a cyclopentyloxy group and a cyclohexyloxy group, a methoxymethyl group, an ethoxymethyl group, an ethoxyethyl group and a butoxymethyl group, a methylamino group, an ethylamino group, a propylamino group, an isopropylamino group, a butylamino group, an isobutylamino group, a sec-butylamino group, a tert-butylamino group, an neopentylamino group, a 1-ethylpropylamino group, a n-pentylamino group, a hexylamino group, a heptylamino group and an octylamino group, an allyl group, an isopropenyl group, a 1-butenyl group, a 2-butenyl group, a 2-pentenyl group and a 5-hexenyl group, a benzyl group and a phenethyl group.

Other preferred compounds of formula (I) according to the invention are those wherein R represents a hydrogen atom, a substituted or non-substituted C₁-C₈-alkyl, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms.

More preferred compounds of formula (I) according to the invention are those wherein R represents a hydrogen atom, a substituted or non-substituted C₁-C₈-alkyl, substituted or non-substituted C₁-C₈-cycloalkyl or substituted or non-substituted C₁-C₈-alkoxy.

The above mentioned preferences with regard to the substituents of the compounds of formula (I) according to the invention can be combined in various manners. These combinations of preferred features thus provide sub-classes of compounds according to the invention. Examples of such sub-classes of preferred compounds according to the invention can combine:
preferred features of A with preferred features of one or more of L¹, Het, X and q;
preferred features of L¹ with preferred features of one or more of A, Het, X and q;
preferred features of Het with preferred features of one or more of A, L¹, X and q;
preferred features of X with preferred features of one or more of A, L¹, Het and q;
preferred features of q with preferred features of one or more of A, L¹, Het and X.

In these combinations of preferred features of the substituents of the compounds according to the invention, the said preferred features can also be selected among the more preferred features of each of A, L¹, Het, X and q; so as to form most preferred subclasses of compounds according to the invention.

The preferred features of the other substituents of the compounds according to the invention can also be part of such sub-classes of preferred compounds according to the invention, notably the groups of substituents Y, n, m, p, R¹, R², Z, Q, L and R.

The present invention also relates to a process for the preparation of compounds of formula (I), Thus, according to a further aspect of the present invention, there is a provided process P1 for the preparation of compounds of formula (I), as herein-defined, as illustrated by the following reaction schemes.

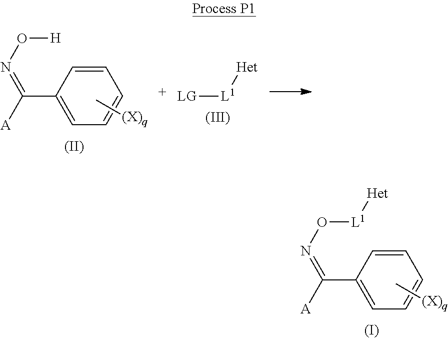

wherein A, X, q, Z, L¹ and Het are as herein-defined and LG represents a leaving group. Suitable leaving groups can be selected in the list consisting of a halogen atom or other to customary nucleofugal groups such as triflate, mesylate or tosylate.

According to the invention, there is provided a further process P2 for the preparation of compounds of formula (Ib) from compounds of formula (Ia).

For the compounds of formula (Ia) according to the invention if Z represents an amino group, process P1 according to the invention can be completed by a further step comprising the additional modification of this group, notably by a reaction of acylation, alkoxycarbonylation or alkylaminocarbonylation to yield to a compound of formula (Ib), according to known methods. In such a case there is provided a process P2 according to the invention and such a process P2 can be illustrated by the following reaction schemes:

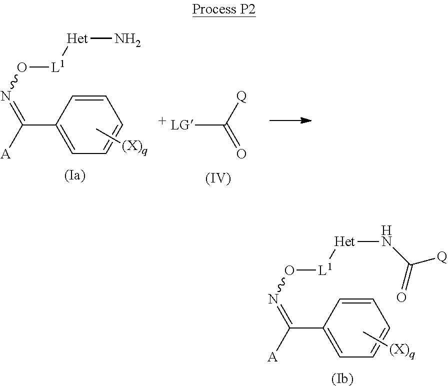

wherein A, X, q, Z, L¹, Q and Het are as herein-defined and LG′ represents a leaving group. Suitable leaving groups can be selected in the list consisting of a halogen atom or other customary nucleofugal groups such as alcoolate, hydroxide or cyanide.

If Z represents a protected amino group, carrying out process P2 would previously require a deprotection step in order to yield the amino group. Amino-protecting groups and related methods of cleavage thereof are known and can be found in T. W. Greene and P. G. M. Wuts, Protective Group in Organic Chemistry, 3^{rd} ed., John Wiley & Sons.

According to the invention, processes P1 and P2 can be performed if appropriate in the presence of a solvent and if appropriate in the presence of a base.

According to the invention, processes P1 and P2 can be performed if appropriate in the presence of a catalyst. Suitable catalyst can be chosen as being 4-dimethyl-aminopyridine, 1-hydroxy-benzotriazole or dimethylformamide.

In case LG′ represents a hydroxy group, the process P2 according to the present invention can be performed in the presence of condensing agent. Suitable condensing agent can be chosen as being acid halide former, such as phosgene, phosphorous tri-bro-mide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl chloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloromethane, 4-(4,6-dimethoxy[1.3.5]triazin-2-yl)-4-methylmorpholinium chloride hydrate or bromo-tripyrrolidino-phosphonium-hexafluorophosphate.

Suitable solvents for carrying out processes P1 and P2 according to the invention are customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl tert-butyl ether, methyl tert-amyl ether, dioxane, tetrahydrofuran, 1,2-dimethoxyethane, 1,2-diethoxyethane or anisole; nitriles, such as acetonitrile, propionitrile, n- or iso-butyronitrile or benzonitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulphoxides, such as dimethyl sulphoxide or sulphones, such as sulpholane.

Suitable bases for carrying out processes P1 and P2 according to the invention are inorganic and organic bases which are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as sodium carbonate, potassium carbonate, potassium bicarbonate, sodium bicarbonate, cesium carbonate, alkali metal or alkaline earth metal acetates, such as sodium acetate, potassium acetate, calcium acetate and also tertiary amines, such as trimethylamine, triethylamine, diisopropylethylamine, tributylamine, N,N-dimethylaniline, pyridine, N-methylpiperidine, N,N-dimethylaminopyridine, 1,4-diazabicyclo[2.2.2]octane (DABCO), 1,5-diazabicyclo[4.3.0]non-5-ene (DBN) or 1,8-diaza-bicyclo[5.4.0]undec-7-ene (DBU).

If carrying out processes P1 and P2 according to the invention, the reaction temperature can independently be varied within a relatively wide range. Generally, process P1 according to the invention is carried out at temperatures between −20° C. and 160° C.

Processes P1 and P2 according to the invention are generally independently carried out under atmospheric pressure. However, it is also possible to operate under elevated or reduced pressure.

If carrying out process P1 according to the invention, generally 1 mol or an excess of derivative of formula A-L¹-LG and from 1 to 3 mol of base are employed per mole of hydroximoyl tetrazole of formula (IV). It is also possible to employ the reaction components in other ratios.

Work-up is carried out by customary methods. Generally, the reaction mixture is treated with water and the organic phase is separated off and, after drying, concentrated under reduced pressure. If appropriate, the remaining residue can be freed by customary methods, such as chromatography or recrystallization, from any impurities that can still be present.

Compounds according to the invention can be prepared according to the above described processes. It will nevertheless be understood that, on the basis of his general knowledge and of available publications, the skilled worker will be able to adapt these processes according to the specifics of each of the compounds according to the invention that is desired to be synthesised. If A represents a group of formula (A¹), as herein-described, the compounds of formula (II), useful as a starting material, can be prepared, for example, by reacting hydroxylamine with the corresponding ketones that can be prepared, for example, according to the method described by R. Raap (Can. J. Chem. 1971, 49, 2139) by addition of a tetrazolyl lithium species to esters of formula

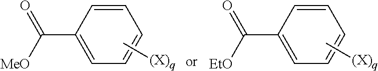

or any of their suitable synthetic equivalents like, for example:

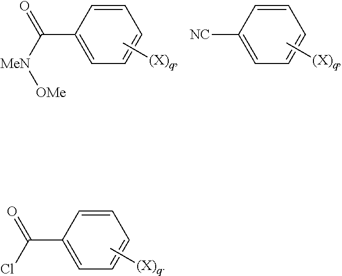

If A represents a group of formula (A²) as herein-described, the compounds of formula (II) useful to as a starting material can be prepared, for example from oximes of formula

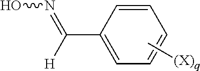

and 5-substituted tetrazole according to the method described by J. Plenkiewicz et al. (Bull. Soc. Chim. Belg. 1987, 96, 675).

In a further aspect, the present invention relates to compounds of formula (II) or compounds of formula (III) useful as intermediate compounds or materials for the process of preparation according to the invention. The present invention thus provides compounds of formula (II) or compounds of formula (III) wherein A, X, q, LG, L¹ and Het are as herein-defined.

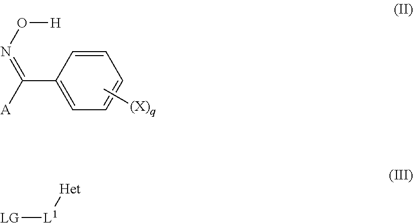

In a further aspect, the present invention also relates to a fungicide composition comprising an effective and non-phytotoxic amount of an active compound of formula (I).

The expression “effective and non-phytotoxic amount” means an amount of composition according to the invention which is sufficient to control or destroy the fungi present or liable to appear on the crops and which does not entail any appreciable symptom of phytotoxicity for the said crops. Such an amount can vary within a wide range depending on the fungus to be controlled, the type of crop, the climatic conditions and the compounds included in the fungicide composition according to the invention. This amount can be determined by systematic field trials, which are within the capabilities of a person skilled in the art.

Thus, according to the invention, there is provided a fungicide composition comprising, as an active ingredient, an effective amount of a compound of formula (I) as herein defined and an agriculturally acceptable support, carrier or filler.

According to the invention, the term “support” denotes a natural or synthetic organic or inorganic compound with which the active compound of formula (I) is combined or associated to make it easier to apply, notably to the parts of the plant. This support is thus generally inert and should be agriculturally acceptable. The support can be a solid or a liquid. Examples of suitable supports include clays, natural or synthetic silicates, silica, resins, waxes, solid fertilisers, water, alcohols, in particular butanol organic solvents, mineral and plant oils and derivatives thereof. Mixtures of such supports can also be used.

The composition according to the invention can also comprise additional components. In particular, the composition can further comprise a surfactant. The surfactant can be an emulsifier, a dispersing agent or a wetting agent of ionic or non-ionic type or a mixture of such surfactants. Mention can be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols and derivatives of the above compounds containing sulphate, sulphonate and phosphate functions. The presence of at least one surfactant is generally essential if the active compound and/or the inert support are water-insoluble and if the vector agent for the application is water. Preferably, surfactant content can be comprised from 5% to 40% by weight of the composition.

Optionally, additional components can also be included, e.g. protective colloids, adhesives, thickeners, thixotropic agents, penetration agents, stabilisers, sequestering agents. More generally, the active compounds can be combined with any solid or liquid additive, which complies with the usual formulation techniques.

In general, the composition according to the invention can contain from 0.05 to 99% by weight of active compound, preferably 10 to 70% by weight.

Compositions according to the invention can be used in various forms such as aerosol dispenser, capsule suspension, cold fogging concentrate, dustable powder, emulsifiable concentrate, emulsion oil in water, emulsion water in oil, encapsulated granule, fine granule, to flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. These compositions include not only compositions which are ready to be applied to the plant or seed to be treated by means of a suitable device, such as a spraying or dusting device, but also concentrated commercial compositions which must be diluted before application to the crop.

The compounds according to the invention can also be mixed with one or more insecticide, fungicide, bactericide, attractant, acaricide or pheromone active substance or other compounds with biological activity. The mixtures thus obtained have a broadened spectrum of activity. The mixtures with other fungicide compounds are particularly advantageous. The composition according to the invention comprising a mixture of a compound of formula (I) with a bactericide compound can also be particularly advantageous

According to another object of the present invention, there is provided a method for controlling the phytopathogenic fungi of plants, crops or seeds, characterized in that an agronomically effective and substantially non-phytotoxic quantity of a pesticide composition according to the invention is applied as seed treatment, foliar application, stem application, drench or drip application (chemigation) to the seed, the plant or to the fruit of the plant or to soil or to inert substrate (e.g. inorganic substrates like sand, rockwool, glasswool; expanded minerals like perlite, vermiculite, zeolite or expanded clay), Pumice, Pyroclastic materials or stuff, synthetic organic substrates (e.g. polyurethane) organic substrates (e.g. peat, composts, tree waste products like coir, wood fibre or chips, tree bark) or to a liquid substrate (e.g. floating hydroponic systems, Nutrient Film Technique, Aeroponics) wherein the plant is growing or wherein it is desired to grow.

The expression “are applied to the plants to be treated” is understood to mean, for the purposes of the present invention, that the pesticide composition which is the subject of the invention can be applied by means of various methods of treatment such as:
spraying onto the aerial parts of the said plants a liquid comprising one of the said compositions,
dusting, the incorporation into the soil of granules or powders, spraying, around the said plants and in the case of trees injection or daubing,
coating or film-coating the seeds of the said plants with the aid of a plant-protection mixture comprising one of the said compositions.

The method according to the invention can either be a curing, preventing or eradicating method. In this method, a composition used can be prepared beforehand by mixing the two or more active compounds according to the invention.

According to an alternative of such a method, it is also possible to apply simultaneously, successively or separately compounds (A) and (B) so as to have the conjugated (A)/(B) effects, of distinct compositions each containing one of the two or three active ingredients (A) or (B).

The dose of active compound usually applied in the method of treatment according to the invention is generally and advantageously
for foliar treatments: from 0.1 to 10,000 g/ha, preferably from 10 to 1,000 g/ha, more preferably from 50 to 300 g/ha; in case of drench or drip application, the dose can even be reduced, especially while using inert substrates like rockwool or perlite;
for seed treatment: from 2 to 200 g per 100 kilogram of seed, preferably from 3 to 150 g per 100 kilogram of seed;
for soil treatment: from 0.1 to 10,000 g/ha, preferably from 1 to 5,000 g/ha.

The doses herein indicated are given as illustrative Examples of method according to the invention. A person skilled in the art will know how to adapt the application doses, notably according to the nature of the plant or crop to be treated.

Under specific conditions, for example according to the nature of the phytopathogenic fungus to be treated or controlled, a lower dose can offer adequate protection. Certain climatic conditions, resistance or other factors like the nature of the phytopathogenic fungi or the degree of infestation, for example, of the plants with these fungi, can require higher doses of combined active ingredients. The optimum dose usually depends on several factors, for example on the type of phytopathogenic fungus to be treated, on the type or level of development of the infested plant, on the density of vegetation or alternatively on the method of application.

Without it being limiting, the crop treated with the pesticide composition or combination according to the invention is, for example, grapevine, but this could be cereals, vegetables, lucerne, soybean, market garden crops, turf, wood, tree or horticultural plants.

The method of treatment according to the invention can also be useful to treat propagation material such as tubers or rhizomes, but also seeds, seedlings or seedlings pricking out and plants or plants pricking out. This method of treatment can also be useful to treat roots. The method of treatment according to the invention can also be useful to treat the over-ground parts of the plant such as trunks, stems or stalks, leaves, flowers and fruit of the concerned plant. Among the plants that can be protected by the method according to the invention, mention can be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for instance banana trees and plantins), Rubiaceae sp., Theaceae sp., Sterculiceae sp., Rutaceae sp. (for instance lemons oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

The composition according to the invention can also be used in the treatment of genetically modified organisms with the compounds according to the invention or the agrochemical compositions according to the invention. Genetically modified plants are plants into genome of which a heterologous gene encoding a protein of interest has been stably integrated. The expression “heterologous gene encoding a protein of interest” essentially means genes which give the transformed plant new agronomic properties or genes for improving the agronomic quality of the modified plant.

The composition according to the invention can also be used against fungal diseases liable to grow on or inside timber. The term “timber” means all types of species of wood and all types of working of this wood intended for construction, for example solid wood, high-density wood, laminated wood and plywood. The method for treating timber according to the invention mainly consists in contacting one or more compounds according to the invention or a composition according to the invention; this includes for example direct application, spraying, dipping, injection or any other suitable means.

Among the diseases of plants or crops that can be controlled by the method according to the to invention, mention can be made of:

Powdery mildew diseases such as:
Blumeria diseases, caused for example by Blumeria graminis;
Podosphaera diseases, caused for example by Podosphaera leucotricha;
Sphaerotheca diseases, caused for example by Sphaerotheca fuliginea;
Uncinula diseases, caused for example by Uncinula necator;

Rust diseases such as:
Gymnosporangium diseases, caused for example by Gymnosporangium sabinae;
Hemileia diseases, caused for example by Hemileia vastatrix;
Phakopsora diseases, caused for example by Phakopsora pachyrhizi or Phakopsora meibomiae;
Puccinia diseases, caused for example by Puccinia recondite;
Uromyces diseases, caused for example by Uromyces appendiculatus;

Oomycete diseases such as:
Bremia diseases, caused for example by Bremia lactucee;
Peronospora diseases, caused for example by Peronospora pisi or P. brassicae;
Phytophthora diseases, caused for example by Phytophthora infestans;
Plasmopara diseases, caused for example by Plasmopara viticola;
Pseudoperonospora diseases, caused for example by Pseudoperonospora humuli or Pseudoperonospora cubensis;
Pythium diseases, caused for example by Pythium ultimum;

Leafspot, leaf blotch and leaf blight diseases such as:
Alternaria diseases, caused for example by Alternaria solani;
Cercospora diseases, caused for example by Cercospora beticola;
Cladiosporum diseases, caused for example by Cladiosporium cucumerinum;
Cochliobolus diseases, caused for example by Cochliobolus sativus;
Colletotrichum diseases, caused for example by Colletotrichum lindemuthanium;
Cycloconium diseases, caused for example by Cycloconium oleaginum;
Diaporthe diseases, caused for example by Diaporthe citri;
Elsinoe diseases, caused for example by Elsinoe fawcettii;
Gloeosporium diseases, caused for example by Gloeosporium laeticolor;
Glomerella diseases, caused for example by Glomerella cingulata;
Guignardia diseases, caused for example by Guignardia bidwelli;
Leptosphaeria diseases, caused for example by Leptosphaeria maculans; Leptosphaeria nodorum;
Magnaporthe diseases, caused for example by Magnaporthe grisea;
Mycosphaerella diseases, caused for example by Mycosphaerella graminicola; Mycosphaerella arachidicola; Mycosphaerella fijiensis;
Phaeosphaeria diseases, caused for example by Phaeosphaeria nodorum;
Pyrenophora diseases, caused for example by Pyrenophora teres;
Ramularia diseases, caused for example by Ramularia collo-cygni;
Rhynchosporium diseases, caused for example by Rhynchosporium secalis;
Septoria diseases, caused for example by Septoria apii or Septoria lycopercisi;
Typhula diseases, caused for example by Typhula incamata;
Venturia diseases, caused for example by Venturia inaequalis;

Root and stem diseases such as:
Corticium diseases, caused for example by Corticium graminearum;
Fusarium diseases, caused for example by Fusarium oxysporum;
Gaeumannomyces diseases, caused for example by Gaeumannomyces graminis;
Rhizoctonia diseases, caused for example by Rhizoctonia solani;
Tapesia diseases, caused for example by Tapesia acuformis;
Thielaviopsis diseases, caused for example by Thielaviopsis basicola;

Ear and panicle diseases such as:
Alternaria diseases, caused for example by Alternaria spp.;
Aspergillus diseases, caused for example by Aspergillus flavus;
Cladosporium diseases, caused for example by Cladosporium spp.;
Claviceps diseases, caused for example by Claviceps purpurea;
Fusarium diseases, caused for example by Fusarium culmorum;
Gibberella diseases, caused for example by Gibberella zeae;
Monographella diseases, caused for example by Monographella nivalis;

Smut and bunt diseases such as:
Sphacelotheca diseases, caused for example by Sphacelotheca reiliana;
Tilletia diseases, caused for example by Tilletia caries;
Urocystis diseases, caused for example by Urocystis occulta;
Ustilago diseases, caused for example by Ustilago nuda;

Fruit rot and mould diseases such as:
Aspergillus diseases, caused for example by Aspergillus flavus;
Botrytis diseases, caused for example by Botrytis cinerea;
Penicillium diseases, caused for example by Penicillium expansum;
Sclerotinia diseases, caused for example by Sclerotinia sclerotiorum;
Verticilium diseases, caused for example by Verticilium alboatrum;

Seed and soilborne decay, mould, wilt, rot and damping-off diseases:
Alternaria diseases, caused for example by Alternaria brassicicola
Aphanomyces diseases, caused for example by Aphanomyces euteiches
Ascochyta diseases, caused for example by Ascochyta lentis
Aspergillus diseases, caused for example by Aspergillus flavus
Cladosporium diseases, caused for example by Cladosporium herbarum
Cochliobolus diseases, caused for example by Cochliobolus sativus
(Conidiaform: Drechslera, Bipolaris Syn: Helminthosporium);
Colletotrichum diseases, caused for example by Colletotrichum coccodes;
Fusarium diseases, caused for example by Fusarium culmorum;
Gibberella diseases, caused for example by Gibberella zeae;
Macrophomina diseases, caused for example by Macrophomina phaseolina
Monographella diseases, caused for example by Monographella nivalis;
Penicillium diseases, caused for example by Penicillium expansum
Phoma diseases, caused for example by Phoma lingam
Phomopsis diseases, caused for example by Phomopsis sojae;
Phytophthora diseases, caused for example by Phytophthora cactorum;
Pyrenophora diseases, caused for example by Pyrenophora graminea
Pyricularia diseases, caused for example by Pyricularia oryzae;
Pythium diseases, caused for example by Pythium ultimum;
Rhizoctonia diseases, caused for example by Rhizoctonia solani;
Rhizopus diseases, caused for example by Rhizopus oryzae
Sclerotium diseases, caused for example by Sclerotium rolfsii;
Septoria diseases, caused for example by Septoria nodorum;
Typhula diseases, caused for example by Typhula incarnata;
Verticillium diseases, caused for example by Verticillium dahliae;

Canker, broom and dieback diseases such as:
Nectria diseases, caused for example by Nectria gaffigena;

Blight diseases such as:
Monilinia diseases, caused for example by Monilinia laxa;

Leaf blister or leaf curl diseases such as:
Taphrina diseases, caused for example by Taphrina deformans;

Decline diseases of wooden plants such as:
Esca diseases, caused for example by Phaemoniella clamydospora;
Eutypa dyeback, caused for example by Eutypa late;
Dutch elm disease, caused for example by Ceratocystsc ulmi;

Diseases of flowers and Seeds such as:
Botrytis diseases, caused for example by Botrytis cinerea;

Diseases of tubers such as:
Rhizoctonia diseases, caused for example by Rhizoctonia solani
Helminthosporium diseases, caused for example by Helminthosporium solani.

The compounds according to the invention can also be used for the preparation of composition useful to curatively or preventively treat human or animal fungal diseases such as, for example, mycoses, dermatoses, trichophyton diseases and candidiases or diseases caused by Aspergillus spp., for example Aspergillus fumigatus.

The various aspects of the invention will now be illustrated with reference to the following table 1 of compound examples and the following preparation or efficacy examples.

The following table 1 illustrates in a non limiting manner examples of compounds according to the invention.

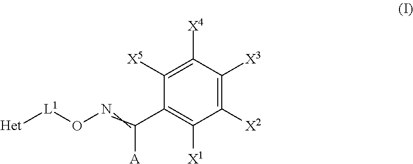

In table 1 we use the following abbreviations for specified claimed elements “Het” and “A” of the generic structure (I) of the invention:

TABLE 1

| Het¹ | | | | A¹ | | | | | | | | | |
|---|---|---|---|---|---|---|---|---|---|---|---|---|---|
| 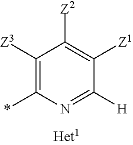 | | | | 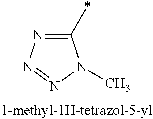 | | | | | | | | | |
| | | | | | | | | | | | | log | MW |
| Ex. | Het | Z¹ | Z² | Z³ | L¹ | X¹ | X² | X³ | X⁴ | X⁵ | A | p | measured |
| 1 | Het¹ | H | H | (phenylacetyl)amino | CH₂ | H | H | H | H | H | A¹ | 2.6 | |
| 2 | Het¹ | H | H | [(pentyloxy)carbonyl]amino | CH₂ | H | H | H | H | H | A¹ | | 424 |
| 3 | Het¹ | H | H | (3-methylbutanoyl)amino | CH₂ | H | H | H | H | H | A¹ | 2.39 | 394 |
| 4 | Het¹ | (phenylacetyl)amino | H | H | CH₂ | H | H | H | H | H | A¹ | 2.93 | 428 |
| 5 | Het¹ | (2,2-dimethylpropanoyl)amino | H | H | CH₂ | H | H | H | H | H | A¹ | 2.51 | 393 |
| 6 | Het¹ | [(pentyloxy)carbonyl]amino | H | H | CH₂ | H | H | H | H | H | A¹ | 3.37 | 424 |
| 7 | Het¹ | (3-methylbutanoyl)amino | H | H | CH₂ | H | H | H | H | H | A¹ | 2.47 | 394 |
| 8 | Het¹ | hexanoylamino | H | H | CH₂ | H | H | H | H | H | A¹ | 2.9 | 408 |
| 9 | Het¹ | H | H | pentanoylamino | CH₂ | H | H | H | H | H | A¹ | 2.44 | 394 |
| 10 | Het¹ | H | H | hexanoylamino | CH₂ | H | H | H | H | H | A¹ | 2.78 | 408 |
| 11 | Het¹ | pentanoylamino | H | H | CH₂ | H | H | H | H | H | A¹ | 2.54 | 394 |
| 12 | Het¹ | amino | H | H | CH₂ | H | H | H | H | H | A¹ | | |
| 13 | Het¹ | H | H | 1,3-dioxo-1,3-dihydro-2H- | CH₂ | H | H | H | H | H | A¹ | 2.63 | 440 |
| | | | | isoindol-2-yl | | | | | | | | | |
| 14 | Het¹ | H | H | amino | CH₂ | H | H | H | H | H | A¹ | 1.01 | 310 |
| 15 | Het¹ | H | amino | H | CH₂ | H | H | H | H | H | A¹ | 1.03 | 310 |
| 16 | Het¹ | 1,3-dioxo-1,3-dihydro-2H- | H | H | CH₂ | H | H | H | H | H | A¹ | 2.82 | 440 |
| | | isoindol-2-yl | | | | | | | | | | | |
| 17 | Het¹ | H | 1,3-dioxo-1,3-dihydro- | H | CH₂ | H | H | H | H | H | A¹ | 2.9 | |
| | | | 2H-isoindol-2-yl | | | | | | | | | | |

Measurement of logP values was performed according EEC directive 79/831 Annex V.A8 by HPLC (High Performance Liquid Chromatography) on reversed phase columns with the following method: Measurement of LC-MS was done at pH 2.7 with 0.1% formic acid in water and with acetonitrile (contains 0.1% formic acid) as eluent with a linear gradient from 10% acetonitrle to 95% acetonitrile.

Calibration was done with not branched alkan2-ones (with 3 to 16 carbon atoms) with known logP-values (measurement of logP values using retention times with linear interpolation between successive alkanones). lambda-maX-values were determined using UV-spectra from 200 nm to 400 nm and the peak values of the chromatographic signals.

In table 1, M+H (or M H) means the molecular ion peak, plus or minus 1 a.m.u. (atomic mass unit) respectively, as observed in mass spectroscopy and M (Apcl+) means the molecular ion peak as it was found via positive atmospheric pressure chemical ionisation in mass spectroscopy.

In all examples of table 1 the M+1 peak was measured.

### Double Bond Geometry:

In the following list we specify the double bond geometry of the examples of table 1 as shown here:

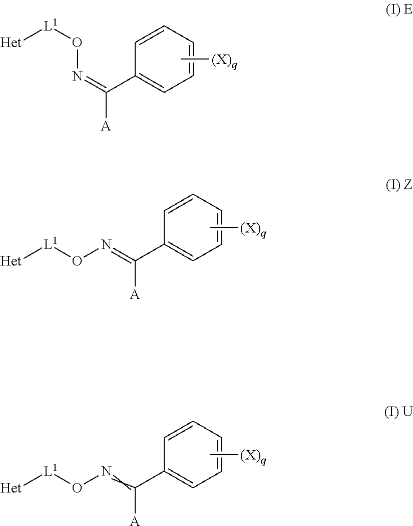

### Example (Double Bond Geometry) of the Examples of Table 1:

All examples of table 1 are of Type “U”.

### EXAMPLE A

### Phytophthora Test (Tomato)/Preventive

Solvent: 49 parts by weight of N,N-Dimethylformamide

Emulsifier: 1 part by weight of Alkylarylpolyglycolether

To produce a suitable preparation of active compound, 1 part by weight of active compound is mixed with the stated amounts of solvent and emulsifier, and the concentrate is diluted with water to the desired concentration.

To test for preventive activity, young plants are sprayed with the preparation of active compound at the stated rate of application. One day after this treatment, the plants are inoculated with an aqueous spore suspension of Phytophthora infestans. The plants remain for one day in an incubation cabinet at approximately 22° C. and a relative atmospheric humidity of 100%. Then the plants are placed in an incubation cabinet at approximately 20° C. and a relative atmospheric humidity of 96%.

The test is evaluated 7 days after the inoculation. 0% means an efficacy which corresponds to that of the control, while an efficacy of 100% means that no disease is observed.

In this test the following compound of table 1 according to the invention showed efficacy of 70% or even higher at a concentration of 500 ppm of active ingredient. Example Nr. 2.

### EXAMPLE B

### Plasmopara Test (Grapevines)/Protective

Solvent: 24.5 parts by weight of acetone
24.5 parts by weight of dimethylacetamide

Emulsifier: 1 part by weight of alkylaryl polyglycol ether

To produce a suitable preparation of active compound, 1 part by weight of active compound is mixed with the stated amounts of solvent and emulsifier, and the concentrate is diluted with water to the desired concentration.

To test for protective activity, young plants are sprayed with the preparation of active compound at the stated rate of application. After the spray coating has dried on, the plants are inoculated with an aqueous spore suspension of Plasmopara viticola and then remain for 1 day in an incubation cabinet at approximately 20° C. and a relative atmospheric humidity of 100%. The plant is subsequently placed for 4 days in a greenhouse at approximately 21° C. and a relative atmospheric humidity of approximately 90%. The plants are then misted and placed for 1 day in an incubation cabinet.

The test is evaluated 6 days after the inoculation. 0% means an efficacy which corresponds to that of the control, while an efficacy of 100% means that no disease is observed.

In this test the following compound of table 1 according to the invention showed efficacy of 70% or even higher at a concentration of 100 ppm of active ingredient: Example Nr. 2.

### EXAMPLE C

### In Vitro-Test for the Calculation of the ED50-Value with Pythium Aphanidermatum

Wells of 96-hole microtitre plates are filled with 10 μl of a solution of the test compound in methanol together with the emulsifier alkylaryl polyglycol ether. Thereafter, the solvent is evaporated in a hood. At the next step, into each well 200 μl of liquid potato dextrose medium is given that has been amended with an appropriate concentration of spores or mycelium suspension of the test fungus. The resulting concentrations of the test compounds in the microtitre well are 50, 5, 0.5 and 0.05 ppm. The resulting concentration of the emulsifier in all wells is constantly 300 ppm. With the aid of a photometer the extinction in all wells is measured at the wavelength of 620 nm.

The microtiter plates are then transferred for 3-5 days onto a shaker at 20° C. and 85% relative humidity.

At the end of the incubation time the growth of the test organisms is measured again photometrically at the wavelength of 620 nm. The difference between the two extinction values (taken before and after incubation) is proportional to the growth of the test organism. Based on the Δ extinction data from the different test concentrations and that of the untreated test organism (control) a dose-response curve is calculated. The concentration that is necessary to give 50% growth inhibition is defined and reported as ED50-value (=Effective Dose that causes 50% growth inhibition) in ppm (=mg/I).

In this test the following compounds of table 1 according to the invention of the following structure showed an ED50-value lower than 1 ppm: Examples Nr. 1, 2, 3, 4, 5, 6, 8, 10, 11 and 13.

The following examples illustrate in a non-limiting manner the preparation and efficacy of the compounds of formula (I) according to the invention.

### Preparation of 2-[({[(1-methyl-1H-tetrazol-5-yl)(phenyl)methylene]amino}oxy)methyl]pyridin-3-amine (Compound 14) according to process P1

### Step 1

### Preparation of 2-{2-[({[(1-methyl-1H-tetrazol-5-yl)(phenyl)methylene]amino}oxy)methyl]pyridin-3-yl}-1H-isoindole-1,3(2H)-dione (Compound 13)

To a stirred solution of N-hydroxy-1-(1-methyl-1H-tetrazol-5-yl)-1-phenylmethanimine (3.0 g, 14.76 mmol, 1 eq.) in 30 ml of acetonitrile, was added potassium iodide (0.49 g, 2.95 mmol, 0.2 eq.), then was added cesium carbonate (5.77 g, 15.50 mmol, 1.2 eq.), and then was added YYYYY (4.91 g, 15.50 mmol, 1.05 eq.). The reaction mixture was stirred for further 24 hrs at room temperature. After evaporation of the solvent, the residue was taken up with 70 ml of water, diluted with ethyl acetate (50 ml). The aqueous layer was separated and extracted with ethyl acetate (50 ml). The organics were combined, dried over MgSO4 and concentrated. The crude material was purified by chromatography on silica gel to give 2-{2-[({[(1-methyl-1H-tetrazol-5-yl)(phenyl)methylene]amino}oxy)methyl]pyridin-3-yl}-1H-isoindole-1,3(2H)-dione (4.62 g, 66% yield) as a white solid.

HPLC/MS: m/z=440 (M+H); logP_{(HCOOH)}=2.63

### Step 2

### Preparation of 2-[({[(1-methyl-1H-tetrazol-5-yl)(phenyl)methylene]amino}oxy)methyl]pyridin-3-amine (Compound 14)

To a stirred solution 2-{2-[({[(1-methyl-1H-tetrazol-5-yl)(phenyl)methylene]amino}oxy)methyl]pyridin-3-yl}-1H-isoindole-1,3(2H)— (4.61 g, 10.49 mmol, 1 eq.) in 100 ml of tetrahydrofurane, was added hydrazine hydrate (2.55 ml, 52.45 mmol, 5 eq.). The reaction mixture was stirred for further 24 hrs at room temperature. The white precipitate which formed was filtered, rinsed twice with 25 ml of tetrahydrofurane. The filtrate was concentrated yielding to 2-[({[(1-methyl-1H-tetrazol-5-yl)(phenyl)methylene]amino}oxy)methyl]pyridin-3-amine (3.00 g, 95% pure, 88% yield).

HPLC/MS: nrilz=310 (M+H); logP_{(HCOOH)}=1.01

### Preparation of pentyl {2-[({[(1-methyl-1H-tetrazol-5-yl)(phenyl)methylene]amino}oxy)methyl]pyridin-3-yl}carbamate (Compound 2) according to process P2

To a stirred solution of 2-[({[(1-methyl-1H-tetrazol-5-yl)(phenyl)methylene]amino}oxy)methyl]pyridin-3-amine (0.12 g, 0.39 mmol, 1 eq.) in 3 ml of acetonitrile, was added pyridine (0.035 ml, 0.43 mmol, 32.6 eq.), then was added (npentyloxy)-carbonyl chloride (0.099 g, 0.66 mmol, 1.7 eq.). The reaction mixture was stirred for 5 hrs, allowed to sit for 48 hrs and filtered through “Chem Elut” cartridge, washed with fresh ethyl acetate (20 ml) The filtrate was evaporated. The crude material was purified by chromatography on silica gel to give pentyl {2-[({[(1-methyl-1H-tetrazol-5-yl)(phenyl)methylene]amino}oxy)methyl]pyridin-3-yl}carbamate (0.074 g, 95% pure, 43% yield) HPLC/MS: m/z=324 (M+H); logP_{(HCOOH)}=3.53

1. A hydroximoyl-tetrazole derivative compound of formula (I): 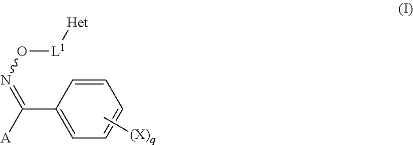
wherein
A represents a tetrazoyl group of formula (A¹) or (A²): 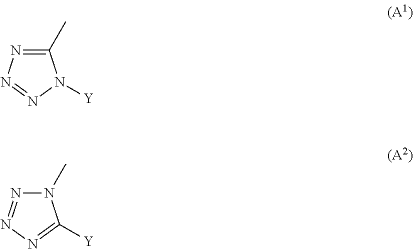
wherein Y represents an alkyl group;
L¹ represents a direct bond or a divalent group selected in the list consisting of
—(CR¹R²)ₙ— —(CR¹R²)ₘ—C(═O)—(CR¹R²)ₚ—
—(CR¹R²)ₘ—(CR¹═CR²)—(CR¹R²)ₚ— —(CR¹R²)ₘ—C(═O)—O—(CR¹R²)ₚ
—(CR¹R²)ₘ—C≡C—(CR¹R²)ₚ— —(CR¹R²)ₘ—O—C(═O)—(CR¹R²)ₚ—
—(CR¹R²)ₘ—O—(CR¹R²)ₚ— —(CR¹R²)ₘ—C(═O)—NH—(CR¹R²)ₚ—
—(CR¹R²)ₘ—NH—(CR¹R²)ₚ— —(CR¹R²)ₘ—NH—C(═O)—(CR¹R²)ₚ—
wherein
n represents 1, 2, 3 or 4;
m and p independently represent 0, 1, 2 or 3;
R¹ and R² are independently selected in the list consisting of hydrogen, halogen, [C₁-C₄]-alkyl, [C₁-C₄]-haloalkyl, [C₂-C₄]-alkenyl, [C₂-C₄]-halo alkenyl, [C₂-C₄]-alkynyl, [C₂-C₄]-haloalkynyl, [C₃-C₅]-cycloalkyl, [C₃-C₅]-halocycloalkyl, [C₁-C₄]-alkoxy, [C₁-C₄]-alkoxy-[C₁-C₄]-alkyl, [C₁-C₄]-alkoxy-[C₁-C₄]-alkoxy, [C₁-C₄]-haloalkoxy, [C₁-C₄]-haloalkoxy-[C₁-C₄]-alkyl, carbonyloxy, [C₁-C₆]-alkoxy-carbonyl and cyano, R¹ and R² can form a 3- to 7-membered, saturated or unsaturated, carbo- or heterocycle;
Het represents a pyridyl group of formula (Het¹) or a thiazolyl group of formula (Het²); 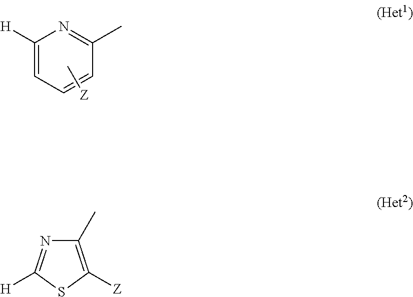
wherein Z represents a an amino group or a group of formula QC(=L)NR— wherein Q represents a hydrogen atom, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy or a substituted or non-substituted phenyl group; L represents an oxygen atom or a sulphur atom; and R represents a hydrogen atom, a substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms;
X independently represents a hydrogen atom, a halogen atom, an alkyl group, an alkoxy group, a cyano group, a methanesulfonyl group, a nitro group, a trifluoromethyl group or an aryl group;
q represents 1, 2, 3, 4 or 5, provided that if q represents 2, 3, 4 or 5 then X represents a halogen atom;
as well as salts, N-oxides, metallic complexes and metalloidic complexes thereof.

2. A compound according to claim 2 wherein Y represents an alkyl group.

3. A compound according to claim 2 wherein Y represents a methyl group, an ethyl group, a n-propyl group or an isopropyl group.

4. A compound according to claim 1 wherein L¹ represents a direct bond or a divalent group selected in the list consisting of
—(CR¹R²)ₙ— —C(═O)—(CR¹R²)ₚ—
—(CR¹R²)ₘ—O— —(CR¹R²)ₘ—C(═O)—O—
—(CR¹R²)ₘ—NH— —(CR¹R²)ₘ—C(═O)—NH—
—(CR¹R²)ₘ—C(═O)— —(CR¹R²)ₘ—NH—C(═O)
wherein
n represents 1 or 2;
m and p independently represent 0 or 1;
R¹ and R² independently represent a hydrogen atom, a halogen atom, a cyano group, substituted or non-substituted C₁-C₈-alkyl, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy, substituted or non-substituted C₃-C₈-halogenoalkynyloxy having 1 to 5 halogen atoms.

5. A compound according to claim 4 wherein L¹ represents a direct bond or a divalent group selected in the list consisting of —(CR¹R²)—, —C(═O)—(CR¹R²)— and —C(═O)—; wherein R¹ and R² are independently selected in the list consisting of hydrogen, halogen, methyl, ethyl, isopropyl, trifluoromethyl, difluoromethyl, allyl, ethynyl, propargyl, cyclopropyl, methoxy, trifluoromethoxy and cyano.

6. A compound according to claim 1 wherein X represents a hydrogen atom, a chlorine atom, a fluorine atom, an alkyl group having 1 to 4 carbon atoms, an alkoxy group having 1 to 3 carbon atoms or a phenyl group, a 4-methylphenyl group or a 4-chlorophenyl group.

7. A compound according to claim 6 wherein X represents a methyl group, an ethyl group, a n-propyl group, an isopropyl group, a n-butyl group, an isobutyl group, a sec-butyl group, a tert-butyl group, a methoxy group, an ethoxy group, a propoxy group and an isopropoxy group.

8. A compound according to claim 1 wherein q represents 1 or 2.

9. A compound according to claim 1 wherein Q represents a hydrogen atom, substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈alkoxy, substituted or non-substituted C₃-C₈-alkynyloxy or a substituted or non-substituted phenyl group.

10. A compound according to claim 9 wherein Q represents a methyl group, an ethyl group, a n-propyl group, an isopropyl group, a 1,1-dimethylpropyl group, a n-butyl group, an isobutyl group, a sec-butyl group, a tert-butyl group, an isoamyl group, a 1-methylbutyl group, a 2-methylbutyl group, an neopentyl group, a 1-ethylpropyl group, a n-pentyl group, a hexyl group, a heptyl group and an octyl group, a chloromethyl group, a difluoromethyl group, a trifluoromethyl group, a difluorochloromethyl group, a pentafluoroethyl group, a 3,3,3-trifluoro-n-propyl group and a 1-chlorohexyl group, a cyclopropyl group, a cyclobutyl group, a cyclopentyl group and a cyclohexyl group, a methoxy group, an ethoxy group, a propoxy group, an isopropoxy group, a 1,1-dimethylpropoxy group, a butoxy group, an isobutoxy group, a sec-butoxy group, a tert-butoxy group, an isopentyloxy group, a 1-methylbutoxy group, a 2-methylbutoxy group, an neopentyloxy group, a 1-ethylpropoxy group, a n-pentyloxy group, a hexyloxy group, a heptyloxy group and an octyloxy group, a cyclopropyloxy group, a cyclobutyloxy group, a cyclopentyloxy group and a cyclohexyloxy group, a methoxymethyl group, an ethoxymethyl group, an ethoxyethyl group and a butoxymethyl group, a methylamino group, an ethylamino group, a propylamino group, an isopropylamino group, a butylamino group, an isobutylamino group, a sec-butylamino group, a tert-butylamino group, an neopentylamino group, a 1-ethylpropylamino group, a n-pentylamino group, a hexylamino group, a heptylamino group and an octylamino group, an allyl group, an isopropenyl group, a 1-butenyl group, a 2-butenyl group, a 2-pentenyl group and a 5-hexenyl group, a benzyl group and a phenethyl group.

11. A compound according to claim 1 wherein R represents a hydrogen atom, a substituted or non-substituted C₁-C₈-alkyl, substituted or non-substituted C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkoxy, substituted or non-substituted C₁-C₈-halogenoalkoxy having 1 to 5 halogen atoms.

12. A compound according to claim 11 wherein R represents a hydrogen atom, a substituted or non-substituted C₁-C₈-alkyl, substituted or non-substituted C₁-C₈-cycloalkyl or substituted or non-substituted C₁-C₈-alkoxy.

13. A compound of formula (II) or (III) 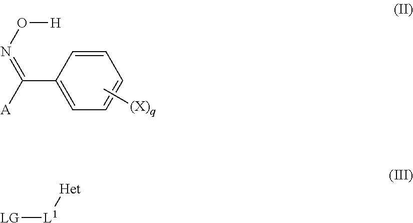
wherein
A represents a tetrazoyl group of formula (A¹) or (A²): 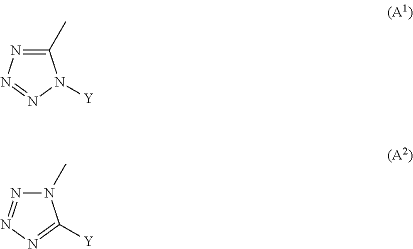
wherein Y represents an alkyl group;
L¹ represents a direct bond or a divalent group selected in the list consisting of
—(CR¹R²)ₙ— —(CR¹R²)ₘ—C(═O)—(CR¹R²)ₚ—
—(CR¹R²)ₘ—(CR¹═CR²)—(CR¹R²)ₚ— —(CR¹R²)ₘ—C(═O)—O—(CR¹R²)ₚ
—(CR¹R²)ₘ—C≡C—(CR¹R²)ₚ— —(CR¹R²)ₘ—O—C(═O)—(CR¹R²)ₚ—
—(CR¹R²)ₘ—O—(CR¹R²)ₚ— —(CR¹R²)ₘ—C(═O)—NH—(CR¹R²)ₚ—
—(CR¹R²)ₘ—NH—(CR¹R²)ₚ— —(CR¹R²)ₘ—NH—C(═O)—(CR¹R²)ₚ—
wherein
n represents 1, 2, 3 or 4;
m and p independently represent 0, 1, 2 or 3;
R¹ and R² are independently selected in the list consisting of hydrogen, halogen, [C₁-C₄]-haloalkyl, [C₁-C₄]-alkenyl, [C₂-C₄]-haloalkenyl, [C₁-C₄]-alkynyl, [C₂-C₄]-haloalkynyl, [C₃-C₅]-cycloalkyl, [C₃-C₅]-halocycloalkyl, [C₁-C₄]-alkoxy, [C₁-C₄]-alkoxy-[C₁-C₄]-alkyl, [C₁-C₄]-alkoxy-[C₁-C₄]-alkoxy, [C₁-C₄]-haloalkoxy, [C₁-C₄]-haloalkoxy-[C₁-C₄]-alkyl, carbonyloxy, [C₁-C₆]-alkoxy-carbonyl and cyano, R¹ and R² can form a 3- to 7-membered, saturated or unsaturated, carbo- or heterocycle;
Het represents a pyridyl group of formula (Het¹) or a thiazolyl group of formula (Het²); 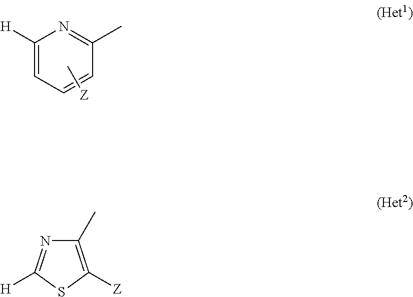
wherein Z represents a an amino group or a group of formula QC(=L)NR— wherein Q represents a hydrogen atom, substituted or non-substituted C₁-C₈-alkyl, substituted or non-substituted tri(C₁-C₈-alkyl)silyl-C₁-C₈-alkyl, substituted or non-substituted C₁-C₈-cyc loalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₂-C₈-alkynyl, substituted or non-substituted C₁-C₈-alkylamino, substituted or non-substituted di-C₁-C₈-alkylamino, substituted or non-substituted C₁-C₈-alkoxy, substituted or non-substituted C₁-C₈-halogenoalkoxy having 1 to 5 halogen atoms, substituted or non-substituted C₁-C₈-alkylsulphenyl, substituted or non-substituted C₁-C₈-halogenoalkylsulphenyl having 1 to 5 halogen atoms, substituted or non-substituted C₂-C₈-alkenyloxy, substituted or non-substituted C₂-C₈-halogenoalkenyloxy having 1 to 5 halogen atoms, substituted or non-substituted C₃-C₈-alkynyloxy or a substituted or non-substituted phenyl group; L represents an oxygen atom or a sulphur atom; and R represents a hydrogen atom, a substituted or non-substituted C₁-C₈-alkyl, substituted or non-substituted tri(C₁-C₈-alkyl)silyl-C₁-C₈-alkyl, substituted or non-substituted C₁-C₈-cycloalkyl, substituted or non-substituted tri(C₁-C₈-alkyl)silyl-C₁-C₈-cycloalkyl, substituted or non-substituted C₁-C₈-halogenoalkyl having 1 to 5 halogen atoms, substituted or non-substituted C₁-C₈-halogenocycloalkyl having 1 to 5 halogen atoms, a C₂-C₈-alkenyl, substituted or non-substituted C₁-C₈-alkynyl, substituted or non-substituted C₁-C₈-alkoxy, substituted or non-substituted C₁-C₈-halogenoalkoxy having 1 to 5 halogen atoms;
X independently represents a hydrogen atom, a halogen atom, an alkyl group, an alkoxy group, a cyano group, a methanesulfonyl group, a nitro group, a trifluoromethyl group or an aryl group;
q represents 1, 2, 3, 4 or 5, provided that if q represents 2, 3, 4 or 5 then X represents a halogen atom;
and LG represents a leaving group.

14. A method for controlling the phytopathogenic fungi or damaging insects of plants, crops or seeds comprising the application of an agronomically effective and substantially non-phytotoxic quantity of the compound according to claim 1 as seed treatment, foliar application, stem application, drench or drip application or chemigation to the seed, the plant or to the fruit of the plant or to soil or to inert substrate, Pumice, Pyroclastic materials or stuff, synthetic organic substrates organic substrates or to a liquid substrate wherein the plant is growing or wherein it is desired to grow.
